(19) United States
(12) Patent Application Publication
(10) Pub. No.: US 2017/0341050 A1
(43) Pub. Date: Nov. 30, 2017

(54) Reaction Method with Homogeneous-Phase Supercritical Fluid and Apparatus for Homogeneous-Phase Supercritical Fluid Reaction

(71) Applicant: NATIONAL SUN YAT-SEN UNIVERSITY, Kaohsiung (TW)

(72) Inventors: Ting-Chang Chang, Kaohsiung (TW); Kuan-Chang Chang, Kaohsiung (TW); Tsung-Ming Tsai, Kaohsiung (TW); Chih-Cheng Shih, Kaohsiung (TW); Chih-Hung Pan, Kaohsiung (TW)

(21) Appl. No.: 15/291,577

(22) Filed: Oct. 12, 2016

(30) Foreign Application Priority Data
May 26, 2016 (TW) 105116469

(51) Int. Cl. B01J 19/00; C07D 473/12; B01J 3/00; H01L 45/00

(52) U.S. Cl. CPC B01J 19/0006; H01L 45/1641; C07D 473/12; B01J 3/008; B01J 2219/00164; B01J 2219/00186

(57) ABSTRACT

The present disclosure provides a reaction method with homogeneous-phase supercritical fluid, including: preparing a supercritical fluid and a solute; supplying the supercritical fluid and the solute into a molecular sieve component to uniformly mix the supercritical fluid and the solute in the molecular sieve component, forming a homogeneous-phase supercritical fluid; and supplying the homogeneous-phase supercritical fluid into a reaction chamber for conducting a reaction. The present disclosure further provides an apparatus for homogeneous-phase supercritical fluid reaction, which can be utilized with the reaction method with homogeneous-phase supercritical fluid

### CROSS REFERENCE TO RELATED APPLICATIONS

The application claims the benefit of Taiwan application serial No. 105116469, filed on May 26, 2016, and the subject matter of which is incorporated herein by reference.

### BACKGROUND OF THE INVENTION

### 1. Field of the Invention

The present invention generally relates to reaction method with supercritical fluid reaction and apparatus for supercritical fluid reaction and, more particularly, to reaction method with homogeneous-phase supercritical fluid and apparatus for homogeneous-phase supercritical fluid reaction.

### 2. Description of the Related Art

During production of resistive memory, a resistive memory is usually placed inside an apparatus for supercritical fluid reaction, and surface treatment is conducted to the resistive memory using a supercritical fluid. A conventional apparatus for supercritical fluid reaction includes a supercritical fluid source, an aqueous solution source and a reaction chamber, with the supercritical fluid source connected to a lateral wall of the reaction chamber via a pipeline, with the liquid source connected to a top wall of the reaction chamber via another pipeline, and with the reaction chamber adapted for receiving the resistive memory and for the surface treatment to be conducted therein. Accordingly, a conventional reaction method with supercritical fluid includes supplying a supercritical fluid and an aqueous solution into the reaction chamber through the pipelines, such that the aqueous solution drops vertically and dissolves in the supercritical fluid which flows horizontally, conducting the surface treatment to the resistive memory at a bottom of the reaction chamber.

However, in the conventional reaction method motioned above, the aqueous solution, as a solute in the supercritical fluid, is mixed with the supercritical fluid by merely gravity force. Hence, the solute and the supercritical fluid cannot be sufficiently mixed together, and the solute tends to be non-uniformly distributed in the supercritical fluid, forming a heterogeneous-phase supercritical fluid. Since the surface treatment to the resistive memory is conducted using such heterogeneous-phase supercritical fluid, the resistive memory presents characteristics with local differences, adversely affecting electrical property and performance of the resultant resistive memory.

In light of the above, it is needed to improve the conventional reaction method with supercritical fluid and apparatus for supercritical fluid reaction.

### SUMMARY OF THE INVENTION

It is therefore the objective of this invention to provide a reaction method with homogeneous-phase supercritical fluid which can uniformly mix a supercritical fluid with a solute, thus forming a homogeneous-phase supercritical fluid for reaction.

The present invention further provides an apparatus for homogeneous-phase supercritical fluid reaction which uniformly mix a supercritical fluid with a solute, thus forming a homogeneous-phase supercritical fluid for reaction.

The present disclosure provides a reaction method with homogeneous-phase supercritical fluid, including: preparing a supercritical fluid and a solute; supplying the supercritical fluid and the solute into a molecular sieve component to uniformly mix the supercritical fluid and the solute in the molecular sieve component, forming a homogeneous-phase supercritical fluid; and supplying the homogeneous-phase supercritical fluid into a reaction chamber for conducting a reaction.

Since the reaction method with homogeneous-phase supercritical fluid of the present disclosure utilizes the molecular sieve component, the solute can be uniformly dissolved in the supercritical fluid, thus forming the homogeneous-phase supercritical fluid. The homogeneous-phase supercritical fluid is provided with a stable, constant solute concentration, thus can be utilized in homogeneous-phase supercritical fluid reaction. Hence, the reaction can be conducted in a uniform manner.

In a form shown, the reaction method with homogeneous-phase supercritical fluid further includes detecting a concentration of the solute in the homogeneous-phase supercritical fluid to obtain a concentration value; and controlling flow rates of the supercritical fluid and the solute into the molecular sieve component based on the concentration value. As such, concentration of the solute can be precisely controlled.

In the form shown, the supercritical fluid is carbon dioxide, alkane, alkene or alcohol. The solute is water or aqueous solution. The molecular sieve component comprises an A-type or X-type molecular sieve. The molecular sieve component comprises molecular sieve material made of aluminum oxide, silicon oxide and stainless steel sintered together. As such, the supercritical fluid, the solute and the molecular sieve can be selected from a large variety to meet different needs.

The present disclosure provides an apparatus for homogeneous-phase supercritical fluid reaction, including: a molecular sieve component having a first inlet, a second inlet and an outlet; a supercritical fluid source connected to the first inlet of the molecular sieve component through a first pipeline; a solute source connected to the second inlet of the molecular sieve component through a second pipeline; and a reaction chamber connected to the outlet of the molecular sieve component through a third pipeline.

Since the apparatus for homogeneous-phase supercritical fluid reaction of the present disclosure includes the molecular sieve component, the solute can be uniformly dissolved in the supercritical fluid, thus forming the homogeneous-phase supercritical fluid. The homogeneous-phase supercritical fluid is provided with a stable, constant solute concentration, thus can be used in a wide variety of applications.

In a form shown, a first valve is arranged on the first pipeline. A second valve is arranged on the second pipeline. A third valve is arranged on the third pipeline. As such, flow rates of the supercritical fluid and the solute flowing into the molecular sieve can be controlled, as well as the homogeneous-phase supercritical fluid flowing into the reaction chamber.

In the form shown, the apparatus for homogeneous-phase supercritical fluid reaction further includes a control component electrically connected with the first valve, the second valve and the third valve. As such, the user can control the first vale, the second valve and the third valve via the control component.

In the form shown, the molecular sieve component further includes an opening. A solute concentration detector is connected to the opening of the molecular sieve component through a fourth pipeline, and the solute concentration detector is electrically connected with the control component. As such, flow rates of the supercritical fluid and the solute flowing into the molecular sieve can be controlled according to the concentration of the solute.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

The sole FIGURE is a schematic diagram of an apparatus for homogeneous-phase supercritical fluid reaction of the present disclosure.

In the various FIGURES of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the invention.

### DETAILED DESCRIPTION OF THE INVENTION

The present disclosure provides a reaction method with homogeneous-phase supercritical fluid, which includes preparing a supercritical fluid and a solute, supplying the supercritical fluid and the solute into the molecular sieve component, such that the supercritical fluid and the solute can be uniformly mixed in the molecular sieve component to form a homogeneous-phase supercritical fluid, and then supplying the homogeneous-phase supercritical fluid into a reaction chamber to conduct a reaction.

Specifically, the supercritical fluid can be water, carbon dioxide, alkane (e.g. methane, ethane, propane, and etc.), alkene (e.g. ethene, propene, and etc.), or alcohol (e.g. methanol, ethanol, propanol, and etc.), which is not limited in the present disclosure. The solute can be any substance that can be dissolved in the supercritical fluid, preferably in liquid state which can be easily mixed with the supercritical fluid. For instance, the solute can be water, ethanol, acetone, oxalic acid, ammonia solution or sulfuric acid. In the present embodiment, the supercritical fluid is carbon dioxide, and the solute is water.

The molecular sieve component can be a vessel filled with molecular sieve material. For instance, the molecular sieve material can be of A-type (e.g. Linde type 3A, 4A or 5A) or X-type (e.g. type 13X), which is not limited in the present disclosure. In the present embodiment, the molecular sieve component comprises molecular sieve material made of aluminum oxide (Al₂O₃), silicon dioxide (SiO₇) and stainless steel sintered together. Since the molecular sieve material is porous, the solute can be absorbed in the pores, thus the concentration of the solute in the homogeneous-phase supercritical fluid can be regulated. Specifically, when the solute concentration increases, the molecular sieve material absorbs the solute to lower its concentration. On the other hand, when the solute concentration decreases, the molecular sieve releases the solute to raise its concentration. Hence, the ratio of the solute to the supercritical fluid can be fixed, providing the homogeneous-phase supercritical fluid with a stable and constant concentration of the solute, thus assuring the later reaction can be conducted in a homogeneous manner.

The homogeneous-phase supercritical fluid can be utilized in a wide variety of reactions, such as surface treatment, cleaning, extraction or solute formation of powder. Accordingly, the reaction chamber can be designed to meet the requirement of the reaction. For instance, when the homogeneous-phase supercritical fluid is used for surface treatment of resistive memory, the reaction chamber can be a chamber for surface treatment and has racks inside for positioning the resistive memory. When the homogeneous-phase supercritical fluid is used for extraction of caffeine, the reaction chamber can be an extraction vessel filled with coffee beans. When the homogeneous-phase supercritical fluid is used for solute formation of powder, the reaction chamber can be an expansion vessel for rapid expansion of the homogeneous-phase supercritical fluid.

Besides, the reaction method with homogeneous-phase supercritical fluid can further include detecting the concentration of the solute in the homogeneous-phase supercritical fluid to obtain a concentration value, and adjusting flow rates of the supercritical fluid and the solute into the molecular sieve based on the concentration value. For instance, the concentration value can be compared with predetermined upper threshold and lower threshold. When the concentration value is higher than the upper threshold, the flow rate of the supercritical fluid can be increased or the flow rate of the solute can be decreased. On the other hand, when the concentration value is lower than the lower threshold, the flow rate of the supercritical fluid can be decreased, or the flow rate of the solute can be increased.

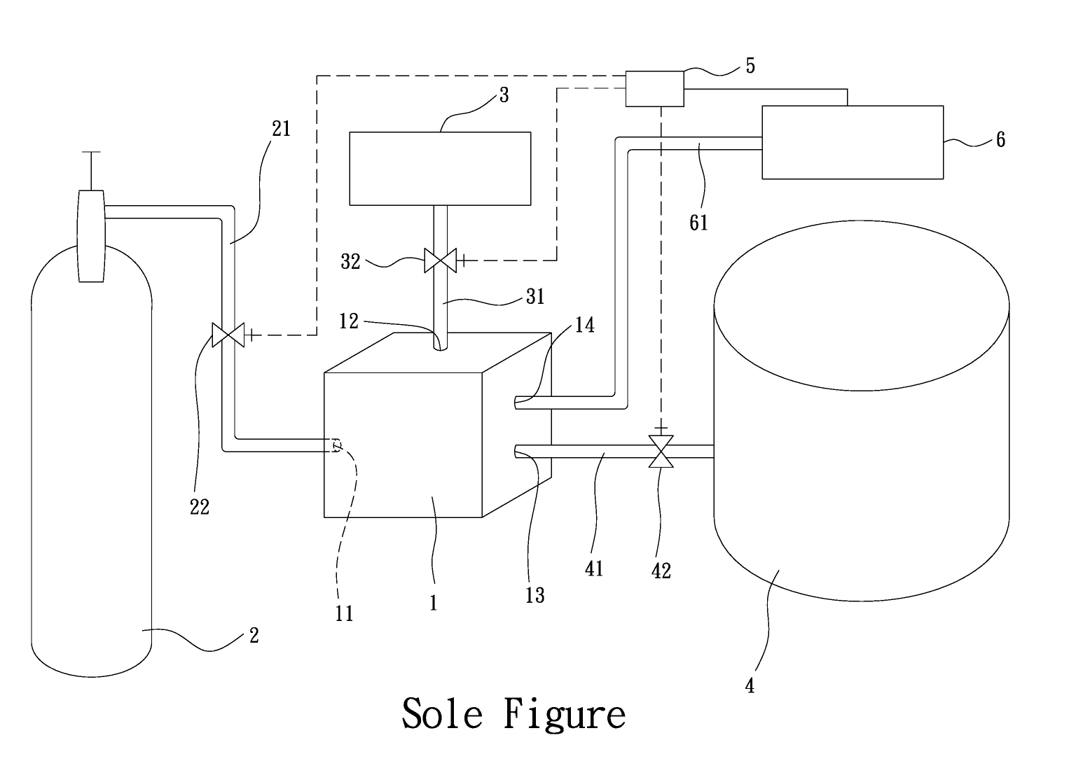

The present invention further provides an apparatus for homogeneous-phase supercritical fluid reaction which can be utilized with the method described above. With references to FIG. 1, the apparatus for homogeneous-phase supercritical fluid reaction includes a molecular sieve component 1, a supercritical fluid source 2, a solute source 3 and a reaction chamber 4, with the supercritical source 2, the solute source 3 and the reaction chamber 4 respectively connected to the molecular sieve component 1.

As described above, the molecular sieve component 1 can be a container filled with molecular sieve material. Besides, the molecular sieve component 1 has a first inlet 11, a second inlet 12 and an outlet 13.

The first and second inlet 11 and 12 are respectively adapted for the supercritical fluid and the solute to flow therethrough into the molecular sieve component 1. The supercritical fluid and the solute can thus be uniformly mixed with assistance of the molecular sieve material, thus forming the homogeneous-phase supercritical fluid. And then, the homogeneous-phase supercritical fluid can flow out of the molecular sieve component 1 through the outlet 13.

The supercritical fluid source 2 is adapted for supplying the supercritical fluid. For instance, the supercritical source 2 can be a device producing supercritical fluid or a storage tank of supercritical fluid. The supercritical fluid source 2 is connected to the first inlet 11 of the molecular sieve component 1 via a first pipeline 21. A first valve 22 can be arranged on the first pipeline 21 for controlling the flow rate of the supercritical fluid into the molecular sieve component 1.

The solute source 3 is adapted for supplying the solute. For instance, the solute source 3 can be a storage tank of solute. When the solute is an aqueous solution, the solute source 3 can be a mixing device for producing the aqueous solution. The solute source 3 is connected to the second inlet 12 of the molecular sieve component 1 via a second pipeline 31. A second valve 32 can be arranged on the second pipeline 31 for controlling the flow rate of the solute into the molecular sieve component 1.

The reaction chamber 3 is adapted for the reaction to be conducted therein using the homogeneous-phase supercritical fluid. As previously described, the reaction chamber 3 can be a chamber for surface treatment, an extraction vessel, or an expansion vessel, which is not limited in the present invention. The reaction chamber 3 is connected to the outlet 13 of the molecular sieve component 1 via a third pipeline 41, such that the homogeneous-phase supercritical fluid can flow through the third pipeline 41 into the reaction chamber 4. A third valve 42 can be arranged on the third pipeline 41 for controlling the flow rate of the homogeneous-phase supercritical fluid from the molecular sieve component 1 into the reaction chamber 4.

The apparatus for homogeneous-phase supercritical fluid reaction of the present disclosure can further include a control component 5. The control component 5 can be electrically connected with the first valve 22, the second valve 32 and the third valve 42 for convenient flow rate control. Specifically, the control component 5 can separately control flow rates of fluid passing through the first valve 22, the second valve 32 and the third valve 42, adjusting the flow rates of the supercritical fluid and the solute into the molecular sieve component 1 and the flow rate of the homogeneous-phase supercritical fluid into the reaction chamber 4. By altering the flow rates of the supercritical fluid and the solute into the molecular sieve component 1, the ratio of the solute to the supercritical fluid can be adjusted. Further, the flow rate of the homogeneous-phase supercritical fluid into the reaction chamber 4 can be adjusted according to the requirement of the reaction conducted in the reaction chamber 4.

Besides, the apparatus for homogeneous-phase supercritical fluid reaction of the present disclosure can further include a solute concentration detector 6, and the molecular sieve component 1 can correspondingly include an opening 14. The solute concentration detector 6 is connected to the opening 14 of the molecular sieve component 1 via a fourth pipeline 61, and is electrically connected to the control component 5. The solute concentration detector 6 is adapted for detecting the solute concentration in the homogeneous-phase supercritical fluid to obtain the concentration value, and can be a UV detector or the like. The solute concentration detector 6 submits the concentration value to the control component 5, and then the control component 5 controls the flow rates through the first and second valves 22 and 32 based on the concentration value. Hence, the ratio of the solute to the supercritical fluid can be adjusted with the control method described above. It is note worthy that the opening 14 is preferably adjacent to the outlet 13, such that the concentration value can precisely reflect the concentration of the homogeneous-phase supercritical fluid flowing from the molecular sieve component 1 to the reaction chamber 4.

According to the above, since the reaction method with homogeneous-phase supercritical fluid of the present disclosure utilizes the molecular sieve component, the solute can be uniformly dissolved in the supercritical fluid, thus forming the homogeneous-phase supercritical fluid. The homogeneous-phase supercritical fluid is provided with a stable, constant solute concentration, thus can be utilized in homogeneous-phase supercritical fluid reaction. Hence, the reaction can be conducted in a uniform manner.

Besides, since the apparatus for homogeneous-phase supercritical fluid reaction of the present disclosure includes the molecular sieve component, the solute can be uniformly dissolved in the supercritical fluid, thus forming the homogeneous-phase supercritical fluid. The homogeneous-phase supercritical fluid is provided with a stable, constant solute concentration, thus can be used in a wide variety of applications.

Further, since the apparatus for homogeneous-phase supercritical fluid reaction of the present disclosure includes the solute concentration detector electrically connected with the control component, the flow rates of the supercritical fluid and the solute into the molecular sieve component can be conveniently controlled based on the concentration value detected by the concentration detector.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A reaction method with homogeneous-phase supercritical fluid, comprising:
preparing a supercritical fluid and a solute;
supplying the supercritical fluid and the solute into a molecular sieve component to uniformly mix the supercritical fluid and the solute in the molecular sieve component, forming a homogeneous-phase supercritical fluid; and
supplying the homogeneous-phase supercritical fluid into a reaction chamber for conducting a reaction.

2. The reaction method with homogeneous-phase supercritical fluid as claimed in claim 1, further comprising detecting a concentration of the solute in the homogeneous-phase supercritical fluid to obtain a concentration value; and controlling flow rates of the supercritical fluid and the solute into the molecular sieve component based on the concentration value.

3. The reaction method with homogeneous-phase supercritical fluid as claimed in claim 1, wherein the supercritical fluid is carbon dioxide, alkane, alkene or alcohol.

4. The reaction method with homogeneous-phase supercritical fluid as claimed in claim 1, wherein the solute is water or aqueous solution.

5. The reaction method with homogeneous-phase supercritical fluid as claimed in claim 1, wherein the molecular sieve component comprises an A-type or X-type molecular sieve.

6. The reaction method with homogeneous-phase supercritical fluid as claimed in claim 1, wherein the molecular sieve component comprises molecular sieve material made of aluminum oxide, silicon oxide and stainless steel sintered together.

7. An apparatus for homogeneous-phase supercritical fluid reaction, comprising:
a molecular sieve component having a first inlet, a second inlet and an outlet;
a supercritical fluid source connected to the first inlet of the molecular sieve component through a first pipeline;
a solute source connected to the second inlet of the molecular sieve component through a second pipeline; and
a reaction chamber connected to the outlet of the molecular sieve component through a third pipeline.

8. The apparatus for homogeneous-phase supercritical fluid reaction as claimed in claim 7, wherein a first valve is arranged on the first pipeline, wherein a second valve is arranged on the second pipeline, and wherein a third valve is arranged on the third pipeline.

9. The apparatus for homogeneous-phase supercritical fluid reaction as claimed in claim 8, further comprising a control component electrically connected with the first valve, the second valve and the third valve.

10. The apparatus for homogeneous-phase supercritical fluid reaction as claimed in claim 9, wherein the molecular sieve component further comprises an opening, wherein a solute concentration detector is connected to the opening of the molecular sieve component through a fourth pipeline, and wherein the solute concentration detector is electrically connected with the control component.
